(19) United States
(12) Patent Application Publication
(10) Pub. No.: US 2009/0137497 A1
(43) Pub. Date: May 28, 2009

(54) ACTIVE INGREDIENT COMBINATIONS OF GLUCOSYL GLYCERIDES AND CREATINE AND/OR CREATININE

(75) Inventors: Thomas Blatt, Wedel (DE); Ute Breitenbach, Hamburg (DE); Christopher Mummert, Bienenbuettel (DE); Thomas Raschke, Pinneberg (DE); Cathrin Scherner, Hamburg (DE); Jens Schulz, Schenefeld (DE); Franz Staeb, Echem (DE)

Correspondence Address: GREENBLUM & BERNSTEIN, P.L.C., 1950 ROLAND CLARKE PLACE, RESTON, VA, 20191, US

(73) Assignee: Beiersdorf AG, Hamburg (DE)

(21) Appl. No.: 11/944,520

(22) Filed: Nov. 23, 2007

(51) Int. Cl. A61K 31/7028; A61K 8/60; A61P 17/00; A61Q 19/00

(52) U.S. Cl. 514/25

(57) ABSTRACT

Active ingredient combinations of one or more glucosyl glycerides and one or more substances selected from creatine, creatinine, creatine phosphate, creatine sulfate, creatine acetate, creatine ascorbate, and creatine derivatives whose carboxyl group is esterified by a mono- or polyfunctional alcohol, and cosmetic preparations comprising these active ingredient combinations.

The present invention relates to active ingredient combinations of glucosyl glycerides and surface-active glucose derivatives and the use thereof in the field of cosmetic as well as pharmaceutical dermatology.

In particular the present invention relates to active ingredients and cosmetic or dermatological preparations containing such active ingredient combinations.

In particular the present invention relates to cosmetic preparations with a content of substances that increase the moisture of the skin.

The outer layer of the epidermis, the stratum corneum (horny layer), is of special significance as important barrier layer among other things for protection against environmental influences and dryness. The horny layer is constantly worn in contact with the environment and must therefore constantly be renewed.

The skin model that is currently recognized in the specialist field describes the horny layer as a two-component system, similar to a brick wall (bricks and mortar model). In this model, the corneocytes (horny cells) correspond to the bricks, and the lipid membrane in the intercellular spaces, which is of complex composition, corresponds to the mortar.

Apart from their barrier action against external chemical and physical influences, the epidermal lipids also contribute to the coherence of the horny layer and have an effect on the smoothness of the skin. In contrast to the sebaceous gland lipids, which do not form a continuous film on the skin, the epidermal lipids are distributed over the entire horny layer.

The extremely complex interaction of the moisture-binding substances and of the lipids of the upper layers of the skin is very important for the regulation of skin moisture. For this reason, cosmetics generally comprise, in addition to balanced lipid mixtures and water, water-binding substances. These include polyols such as glycerol, sorbitol and xylitol, ethoxylated polyols, and hydrolyzed proteins. Use is also made of the substances present in the natural moisturizing factor (NMF) such as, for example, urea, carbohydrates (e.g., glucose) and amino acids (e.g., serine). These substances are therefore of particular importance for the care function of a cosmetic product, for reasons including, in particular, their relatively good compatibility with the skin and mucous membranes.

A problem which remains unresolved, however, is that even the substances glucose and glycerol, which in principle are entirely unobjectionable, may give rise in particularly sensitive individuals and at very high levels to certain symptoms of irritation to the skin and mucous membranes. The aim was therefore to find moisture-providing substances (so-called “moisturizers”) which possess an even better compatibility than, for example, glucose and glycerol.

The advantageous prophylactic and therapeutic effect of creatine in cosmetic and medical skin care is known per se. Creatine (from the Greek: τo κρεασ=“the meat”) is characterized by the following structure:

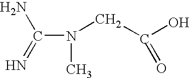

Creatine is found in the myoserum of vertebrates in amounts of 0.05-0.4%, in small amounts also in the brain and blood. As a monohydrate, it is a colorless, crystalline powder. In aqueous solution, creatinine is formed. In the organism, it is formed by the transamidination of L-arginine on glycine to afford guanidinoacetic acid, and subsequent methylation thereof by means of S-adenosyl methionine (by guanidinoacetate methyltransferase). Creatine is regarded as an appetite-promoting constituent of beef and meat extract. The addition of creatine to the diet enhances physical performance.

The prior art is extensive on the cosmetic and dermatological uses of creatine.

Thus, DE 100 32 964 describes the use of creatine and/or creatine derivatives in cosmetic or dermatological preparations for the treatment and prophylaxis of the symptoms of UV-induced and/or ozone-induced skin damage and of inflammatory and degenerative skin conditions.

JP 2000/247866 describes skin cosmetics with a content of creatine and/or creatinine which can be used as a cream or as a milky lotion, where excellent skin care properties are attributed to the relevant preparations.

Furthermore, WO 00/33787 describes the use of creatinine as an effective constituent of deodorants.

Moreover, EP-A-565 010 describes hair growth and hair dye preparations with a content of creatinine phosphate.

Finally, U.S. Pat. No. 4,590,067 and EP-A-178 602 describe the use of creatine or creatinine to produce preparations with anti-inflammatory effect.

However, there is the disadvantage that in aqueous products creatine and creatinine crystallize easily, whereby crystals with non-cosmetic impression form and the effectiveness of the product is reduced.

The object of the present invention was therefore to find ways of avoiding the disadvantages of the prior art. In particular the effect of the repair of the damage associated with the endogenic, chronological and exogenic aging of the skin and the prophylaxis are to be lasting, sustained and without the risk of side effects.

Another object was to find a form of administration for creatine, which is characterized by a reduced tendency to form creatine crystals.

After all this it was surprising and not predictable that active ingredient combinations of
(i) one or more glucosyl glycerides and
(ii) creatine and/or creatinine and/or one or more creatine derivatives selected from the group creatine phosphate, creatine sulfate, creatine acetate, creatine ascorbate and creatine derivatives whose carboxyl group is esterified by mono or polyfunctional alcohols
   or cosmetic preparations containing such active ingredient combinations, eliminate the disadvantages of the prior art.

It was therefore not foreseeable for one skilled in the art that the active ingredient combinations according to the invention or cosmetic or dermatological preparations containing them
have a better effect as moisturizing agent
have a better effect against skin aging
   achieve a better prophylaxis of:
deficient, sensitive or hypoactive skin conditions or deficient, sensitive or hypoactive conditions of cutaneous appendages;
certain degenerative symptoms of the skin (e.g. flabbiness of the skin, age marks, teleangiectasias, atrophy of epidermal and dermal cell layers, the components of the connective tissue, the rete pegs and capillary vessels of the skin) and/or of the cutaneous appendages;
environmentally-induced (smoking, smog, reactive oxygen species, free radicals) and, in particular, light-induced negative changes to the skin and the cutaneous appendages;
light-induced skin damage;
pigmentation disorders;
itchiness;
stratum corneum disorders;
hair loss and for improved hair growth;
inflammatory skin conditions as well as atopic eczema, seborrhoeic eczema, polymorphous light dermatosis, psoriasis, vitiligo
   than active ingredients, active ingredient combinations and preparations of the prior art.

Glucosyl glycerides having the general formula

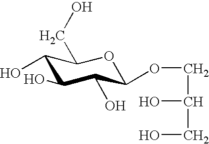

and/or the general formula

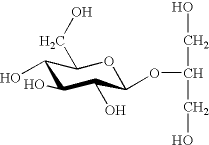

and/or the general formula

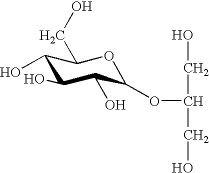

and/or the general formula

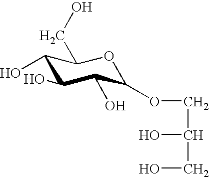

are advantageously preferred according to the present invention.

A particularly preferred glucosyl glyceride is (2-O-β-D-glucopyranosyl)-sn-glycerine.

A particularly advantageous citric acid ester is glyceryl stearate citrate. Such citric acid esters are available, e.g. under the product name “IMWITOR® 370” from Huls AG.

It is advantageous according to the invention to select the molar ratio of one or more glucosyl glycerides to creatine and/or creatinine and/or one or more creatine derivatives selected from the group of creatine phosphate, creatine sulfate, creatine acetate, creatine ascorbate and creatine derivatives whose carboxyl group is esterified by mono- or polyfunctional alcohols from the range of 100:1 to 1:100, preferably 50:1 to 1:50, in particularly preferably 20:1 to 1:20.

According to the use according to the invention, the preparations are particularly advantageously characterized in that creatine and/or creatinine and/or one or more creatine derivatives selected from the group of creatine phosphate, creatine sulfate, creatine acetate, creatine ascorbate and creatine derivatives whose carboxyl group is esterified by mono- or polyfunctional alcohols are present in concentrations of 0.01-10.00% by weight, preferably 0.05-5.00% by weight, particularly preferably 0.1-3.00% by weight, in each case based on the total weight of the preparation.

According to the use according to the invention, the preparations are particularly advantageously characterized in that the glucosyl glyceride(s) are present in concentrations of 0.01-10.00% by weight, preferably 0.05-5.00% by weight, particularly preferably 0.1-3.00% by weight, in each case based on the total weight of the preparation.

According to the use according to the invention, the cosmetic deodorants can be present in the form of aerosols, i.e., preparations that can be sprayed from aerosol containers, squeeze bottles or by a pump device or in the form of liquid preparations that can be applied by means of roll-on devices, as deodorant sticks and in the form of W/O or O/W emulsions, e.g., creams or lotions, that can be applied from normal bottles and containers. Furthermore, the cosmetic deodorants can be advantageously present in the form of deodorizing tinctures, deodorizing feminine hygiene agents, deodorizing shampoos, deodorizing shower or bath preparations, deodorizing powders or deodorizing powder sprays.

In addition to water, ethanol and isopropanol, glycerol and propylene glycol, customary cosmetic carriers which can be employed for preparing the deodorizing formulations according to the use according to the invention are skin-care fat or fat-like substances, such as decyl oleate, cetyl alcohol, cetyl stearyl alcohol and 2-octyl-dodecanol, in the ratios of amounts customary for such preparations, as well as mucilagenous substances and thickeners, for example, hydroxyethyl- or hydroxypropylcellulose, polyacrylic acid, polyvinylpyrrolidone, and in addition also small amounts of cyclic silicone oils (polydimethylsiloxanes), as well as liquid polymethylphenylsiloxanes of low viscosity.

It is likewise advantageous to add conventional antioxidants to the preparations of the present invention. According to the invention, favorable antioxidants which may be used are all antioxidants which are customary or suitable for cosmetic and/or dermatological applications.

The amount of the antioxidants (one or more compounds) in the preparations is preferably 0.001 to 30% by weight, particularly preferably 0.05 to 20% by weight, in particular 1 to 10% by weight, based on the total weight of the preparations.

If the cosmetic or dermatological preparation of the present invention is a solution or emulsion or dispersion, the following can be used as solvents:

water or aqueous solutions
oils, such as triglycerides of capric acid or of caprylic acid, preferably castor oil;
fats, waxes and other natural and synthetic fatty bodies, preferably esters of fatty acids with alcohols of low C number, e.g., with isopropanol, propylene glycol or glycerol, or esters of fatty alcohols with alkanoic acids of low C number or with fatty acids;
alcohols, diols or polyols of low C number, and ethers thereof, preferably ethanol, isopropanol, propylene glycol, glycerol, ethylene glycol, ethylene glycol monoethyl or monobutyl ether, propylene glycol monomethyl, monoethyl or monobutylether, diethylene glycol monomethyl or monoethyl ether and analogous products.

In particular mixtures of the above-mentioned solvents are used. In the case of alcoholic solvents, water can be a further constituent.

If the cosmetic or dermatological preparation of the present invention is a solution or emulsion or dispersion, the following can be used as solvents:
water or aqueous solutions
oils, such as triglycerides of capric acid or of caprylic acid, preferably castor oil;
fats, waxes and other natural and synthetic fatty bodies, preferably esters of fatty acids with alcohols of low C number, e.g., with isopropanol, propylene glycol or glycerol, or esters of fatty alcohols with alkanoic acids of low C number or with fatty acids;
alcohols, diols or polyols of low C number, and ethers thereof, preferably ethanol, isopropanol, propylene glycol, glycerol, ethylene glycol, ethylene glycol monoethyl or monobutyl ether, propylene glycol monomethyl, monoethyl or monobutylether, diethylene glycol monomethyl or monoethyl ether and analogous products.

In particular mixtures of the above-mentioned solvents are used. In the case of alcoholic solvents, water can be a further constituent.

Suitable propellants for cosmetic and/or dermatological formulations according to the invention which can be sprayed from aerosol containers include the customary known readily volatile, liquefied propellants, for example, hydrocarbons (propane, butane, isobutane), which can be employed individually or as mixtures. Compressed air can also advantageously be used.

Of course, one skilled in the art is aware that there are propellant gases which are nontoxic per se and would in principle be suitable for realizing the present invention in the form of aerosol preparations, but which nevertheless should be avoided because of their unacceptable impact on the environment or other accompanying circumstances, in particular fluorocarbons and chlorofluorocarbons (CFCs).

Cosmetic preparations according to the present invention can also be in the form of gels which, in addition to an effective content of the active ingredient according to the invention and solvents which are usually used therefor, preferably water, also contain organic thickeners, e.g., gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, e.g., aluminum silicates, such as, for example, bentonites, or a mixture of polyethylene glycol and polyethylene glycol stearate or distearate. The thickener is present in the gel, e.g., in an amount of between 0.1 and 30% by weight, preferably between 0.5 and 15% by weight.

The following are advantageous exemplary embodiments of the present invention.

### EXAMPLE 1

Gel cream

| | % by weight |
|---|---|
| Acrylate/C₁₀₋₃₀ alkylacrylate crosspolymer | 0.40 |
| Polyacrylic acid | 0.20 |
| Xanthan gum | 0.10 |
| Cetearyl alcohol | 3.00 |
| C12-15 Alkyl benzoate | 4.00 |
| Caprylic/capric triglyceride | 3.00 |
| Cyclic polydimethylsiloxane | 5.00 |
| Dimeticone polydimethylsiloxane | 1.00 |
| Glucosyl glyceride | 4.00 |
| Creatine | 0.50 |
| Creatinine | 0.001 |
| Glycerin | 3.00 |
| Sodium hydroxide | q.s. |
| Preservatives | q.s. |
| Perfume | q.s. |
| Water | ad 100.0 |
| pH value adjusted to 6.0 | |

By mixing the components listed in the table a gel cream was produced of the composition likewise given. The pH value of the gel cream was subsequently adjusted to 6.0.

### EXAMPLE 2

W/O Cream

| | % by weight |
|---|---|
| Polyglyceryl-3-diisostearate | 3.50 |
| Glycerin | 3.00 |
| Polyglyceryl-2-dipolyhydroxystearate | 3.50 |
| Glucosyl glyceride | 4.00 |
| Creatine | 0.60 |
| Creatinine | 0.01 |
| Preservatives | q.s. |
| Perfume | q.s. |
| Magnesium sulfate | 0.6 |
| Isopropyl stearate | 2.0 |
| Caprylylether | 8.0 |
| Cetearyl isononanoate | 6.0 |
| Water | Ad 100.0 |

By mixing the components listed in the table a cream was produced of the composition likewise given on the basis of a water-in-oil dispersion.

1.-6. (canceled)

7. An active ingredient combination of
(i) one or more glucosyl glycerides,
(ii) one or more substances selected from creatine, creatinine, creatine phosphate, creatine sulfate, creatine acetate, creatine ascorbate, and creatine derivatives whose carboxyl group is esterified by a mono- or polyfunctional alcohol.

8. The combination of claim 7, wherein a molar ratio (i):(ii) is from 100:1 to 1:100.

9. The combination of claim 8, wherein the molar ratio (i):(ii) is from 50:1 to 1:50.

10. The combination of claim 8, wherein the molar ratio (i):(ii) is from 20:1 to 1:20.

11. The combination of claim 7, wherein (i) comprises (2-O-β-D-glucopyranosyl)-sn-glycerin.

12. A cosmetic preparation which comprises the active ingredient combination of claim 7.

13. The preparation of claim 12, wherein the preparation comprises from 0.01% to 10.00% by weight of (i), based on a total weight of the preparation.

14. The preparation of claim 13, wherein the preparation comprises from 0.05% to 5.00% by weight of (i).

15. The preparation of claim 13, wherein the preparation comprises from 0.1% to 3.00% by weight of (i).

16. The preparation of claim 12, wherein the preparation comprises from 0.01% to 10.00% by weight of (ii), based on a total weight of the preparation.

17. The preparation of claim 16, wherein the preparation comprises from 0.05% to 5.00% by weight of (ii).

18. The preparation of claim 16, wherein the preparation comprises from 0.1% to 3.00% by weight of (ii).

19. The preparation of claim 12, wherein a molar ratio (i):(ii) is from 100:1 to 1:100.

20. The preparation of claim 19, wherein the molar ratio (i):(ii) is from 50:1 to 1:50.

21. The preparation of claim 19, wherein the molar ratio (i):(ii) is from 20:1 to 1:20.

22. The preparation of claim 12, wherein (i) comprises (2-O-β-D-glucopyranosyl)-sn-glycerin.

23. The preparation of claim 12, wherein the preparation is present as an emulsion.

24. A cosmetic preparation, wherein the cosmetic preparation comprises, based on a total weight of the preparation,
(i) from 0.1% to 3.00% by weight of one or more glucosyl glycerides,
(ii) from 0.1% to 3.00% by weight of one or more substances selected from creatine, creatinine, creatine phosphate, creatine sulfate, creatine acetate, creatine ascorbate, and creatine derivatives whose carboxyl group is esterified by a mono- or polyfunctional alcohol,
a molar ratio (i):(ii) being from 20:1 to 1:20.

25. The preparation of claim 24, wherein (i) comprises (2-O-β-D-glucopyranosyl)-sn-glycerin.

26. A method of caring for skin or skin appendages, wherein the method comprises applying to at least parts of the skin or skin appendages an effective amount of an active ingredient combination of one or more glucosyl glycerides and one or more substances selected from creatine, creatinine, creatine phosphate, creatine sulfate, creatine acetate, creatine ascorbate, and creatine derivatives whose carboxyl group is esterified by a mono- or polyfunctional alcohol.
